(19) United States
(12) Patent Application Publication
(10) Pub. No.: US 2025/0140353 A1
(43) Pub. Date: May 1, 2025

(54) METHOD FOR OPTIMIZING PROPERTIES OF A MOLECULE

(71) Applicant: Samsung Display Co., Ltd., Yongin-si, Gyeonggi-do (KR)

(72) Inventors: Patrick RUOFF, Karlsruhe (DE); Markus SCHMALZL, Bad Schönborn (DE); Matthias SCHNIEWIND, Karlsruhe (DE)

(21) Appl. No.: 18/834,597

(22) PCT Filed: Feb. 10, 2023
(86) PCT No.: PCT/KR2023/002035

§ 371 Date: Jul. 30, 2024

(30) Foreign Application Priority Data
Feb. 11, 2022 (EP) 22156423.0

(51) Int. Cl. G16C 20/50; G16C 20/30; G16C 20/70

(52) U.S. Cl. CPC G16C 20/50; G16C 20/30; G16C 20/70

(57) ABSTRACT

The present invention relates to a computer-implemented method for automated design of a molecule, preferably of an emitter molecule for use in an OLED, in particular of a TADF emitter, the computer-implemented method using a genetic algorithm and as inputs, a set of starting molecular structures including at least one molecular structure, a set of mutation rules for specifying allowed mutations for the genetic algorithm, a scoring function for a molecular structure based on one or more predicted values of the physical and/or chemical properties of the corresponding molecule, and a termination condition, the computer-implemented method providing as output, the molecular structure of the designed molecule.

### CROSS-REFERENCE TO RELATED APPLICATION

The present application is a U.S. National Phase Patent Application of International Patent Application Number PCT/KR2023/002035, filed on Feb. 10, 2023, which claims priority to and the benefit of European Patent Application Number 22156423.0, filed on Feb. 11, 2022, the entire content of each of the two applications is incorporated herein by reference.

### TECHNICAL FIELD

The present technology is related to the field of the design of molecules, especially emitter molecules for the application in OLEDs, and concerns a computer-implemented method for automated design of a molecule, a computer program having program code for performing this method, a method for training of a neural network for use in this method, and a system configured to execute this method and/or the training of such a neural network.

### BACKGROUND ART

Organic electroluminescent devices containing one or more light-emitting layers based on organics such as, e.g., organic light-emitting diodes (OLEDs), light-emitting electrochemical cells (LECs), and light-emitting transistors gain increasing importance. In particular, OLEDs are promising devices for electronic products such as, e.g., screens, displays, and illumination devices. In contrast to most electroluminescent devices essentially based on inorganics, organic electroluminescent devices based on organics are often rather flexible and producible in particularly thin layers.

For many applications, including the design of emitter molecules for OLEDs, molecules with optimized physical and/or chemical properties for their respective application are required. However, due to the high number of theoretically feasible molecular structures, it is typically extremely time-consuming to synthesize a sufficient number of possible molecules and measure the desired values of physical and/or chemical properties and/or simulate the molecules and their properties with methods known from prior art and optimize the molecular structures based on these findings. Hence, there is a need for an improved approach for designing a molecule optimized in regard to its physical and/or chemical properties.

### DISCLOSURE OF INVENTION

### Technical Problem

It is an object of the present invention to provide a method for automated design and optimization of a molecule for use as a material for a certain purpose with regard to the physical and/or chemical properties of the molecule. A physical and/or chemical property of a molecule in the meaning of this invention is to be understood as any physical and/or chemical property either of an individual molecule and/or a (molecular) material consisting of a plurality of these molecules.

### Solution to Problem

An embodiment is a computer-implemented method for automated design of a molecule, preferably of an emitter molecule for use in an OLED, in particular of a TADF emitter. The method using a genetic algorithm and as inputs, a set of starting molecular structures including at least one molecular structure, a set of mutation rules for specifying allowed mutations for the genetic algorithm, a scoring function for a molecular structure based on one or more predicted values of the physical and/or chemical properties of the corresponding molecule, and a termination condition, said method providing as output, the molecular structure of the designed molecule, including the following steps:
a) Providing the set of starting molecular structures as a population of parent molecular structures to the genetic algorithm;
b) Generating a population of offspring molecular structures partially or completely from the population of parent molecular structures using the genetic algorithm by mutating at least one member of the population using the genetic algorithm;
c) Predicting one or more values of the physical and/or chemical properties of the molecules corresponding to the members of the population of offspring molecular structures and calculating a value for the scoring function for each member of the population of offspring molecular structures based thereon,
wherein the one or more values of the physical and/or chemical properties are predicted using a neural network using a transformer with self-attention decay on molecular structures predicting one or more values of the physical and/or chemical properties of the members using the molecular structure as input;
d) Checking if the termination condition is met and if not, generating a new population of parent molecular structures consisting of at least one member of the population of offspring molecular structures having the most optimal value of the scoring function among the offspring molecular structures;
e) Iterating the steps b, c, and d until the termination condition is met;
f) Selecting a molecular structure generated in step b or any of its iterations as an output.

The computer-implemented method is characterized in that the termination condition is met if the scoring function for at least one member of the population offspring structure is greater or smaller than or equal to a pre-defined value and/or a pre-defined number of iterations of the steps b, c, d is reached.

The computer-implemented method is characterized in that the mutating using the genetic algorithm is a fragment-based mutation and/or a string-based mutation.

The computer-implemented method is characterized in that the genetic algorithm uses tournament selection and/or elitism.

The computer-implemented method is characterized in that for the prediction of the one or more values of the physical and/or chemical properties, a computer-implemented method for predicting a value of a physical and/or chemical property of a molecule is used, the method for predicting the value of the physical and/or chemical property using as input

a molecular structure of the molecule as an atom-bond-graph including at least atoms of the molecular structure and bonds of the molecular structure as nodes, the method providing as output, the predicted value of the physical and/or chemical property, including the following steps:
a) Extracting for each node of all n_{nodes} nodes of the atom-bond-graph a feature vector of dimension d_{features}, the feature vector including a node type, the node type preferably being one of atom, bond, and global, and further data on the node in case of the node type being atom or bond;
b) Generating a feature matrix of dimension n_{nodes}×d_{features} composed of the extracted n_{nodes} feature vectors;
c) Calculating a squared distance matrix D of dimension n_{nodes}×n_{nodes} based on and/or including distances between atoms and bonds of the molecular structure;
d) Applying a trained neural network including a transformer using the squared distance matrix D for self-attention decay on the feature matrix to generate a prediction of the value of the physical and/or chemical property of the molecule, wherein the neural network includes:
   a trained input encoder configured to generate an input matrix with dimension n_{nodes}×d_{model} from the feature matrix of dimension n_{nodes}×d_{features}, where d_{model} is the dimension of the transformer model,
   as the transformer, a trained transformer-encoder stack with n_{layers}>1 transformer-encoder layers using the squared distance matrix D for self-attention decay, wherein the trained transformer-encoder stack is configured to generate a matrix of dimension n_{nodes}×d_{model} as output using the input matrix of the input encoder as input,
   a trained projection layer including a self-attention layer configured to generate a vector of dimension d_{model} as output using the matrix generated by the trained transformer-encoder stack as input,
   a trained multilayer perceptron configured to generate the prediction of the value of the physical and/or chemical property from the output vector of the trained projection layer.

The computer-implemented method is characterized in that each of the n_{layers} trained transformer-encoder layers includes a trained multi-head decaying self-attention, a trained feed-forward network, two trained layer normalizations, and two residual connections.

The computer-implemented method is characterized in that an additional input parameter is used as an additional input for the trained multilayer perceptron.

The computer-implemented method is characterized in that the physical and/or chemical property is one of a HOMO energy level, a LUMO energy level, a singlet energy level, a triplet energy level, a singlet-triplet energy gap, an oscillator strength, a dipole moment, a photo-luminescent quantum yield, a delayed fluorescence lifetime, and/or a peak emission wavelength.

The computer-implemented method is characterized in that the squared distance matrix D is calculated by using abstract distances derived from the atom-bond-graph.

The computer-implemented method is characterized in that a part of the trained neural network, for example, the transformer of the trained neural network, was initialized by another transformer of another trained neural network suitable for performing a method according to any one of the preceding embodiments.

An embodiment is a method for training of a neural network for use in the computer-implemented method, wherein the method uses a system including at least one processor and a storage device and the neural network includes a transformer configured to use a squared distance matrix D for attention decay, one or more projection layers including an attention layer configured to generate a vector as output using a matrix generated by the transformer as input, and one or more multilayer perceptrons using the output of one of the one or more projection layers as input, the method including the following steps:
a) Providing a data set including a molecular structure of a molecule and a value of a physical and/or chemical property of the molecule, where the physical and/or chemical property is assigned to one of the multilayer perceptrons;
b) Converting the molecular structure of each molecule to an atom-bond-graph including at least atoms of the molecular structure and bonds of the molecular structure as nodes;
c) Extracting for each node of all n_{nodes} nodes of the atom-bond-graph (100) a feature vector of dimension d_{features}, the feature vector including a node type, the node type preferably being one of atom, bond, and global, and further data on the node in case of the node type being atom or bond;
d) Generating a feature matrix of dimension n_{nodes}×d_{features} composed of the extracted n_{nodes} feature vectors;
e) Calculating the squared distance matrix D of dimension n_{nodes}×n_{nodes} based on and/or including distances between atoms and bonds of the molecular structure;
f) Generating an output value of the multilayer perceptron assigned to the physical and/or chemical property; and
g) Adjusting the neural network based on comparing the output value of the multilayer perceptron to the value of the physical and/or chemical property assigned to the multilayer perceptron.

In the method, the neural network includes at least two multilayer perceptrons characterized in that the steps a to g are repeated for a different data set including the molecular structure of the molecule and a value of a different physical and/or chemical property, the different physical and/or chemical property being assigned to a different multilayer perceptron.

The method is characterized in that the steps a to g are repeated for a different data set including the molecular structure of a different molecule and a value of the physical and/or chemical property of the different molecule.

The method is characterized in that a part of the neural network, for example the transformer, is initialized using another transformer of another trained neural network suitable for use the in computer-implemented method.

A system including a processor and a storage device, wherein the system is configured to execute the methods.

A computer program having a program code for performing the methods.

### Advantageous Effects of Invention

The present invention to provide a method for automated design and optimization of a molecule for use as a material for a certain purpose with regard to the physical and/or chemical properties of the molecule.

### BRIEF DESCRIPTION OF DRAWINGS

Hereinafter, specific embodiments are described referring to the drawings, wherein:

FIG. 1 shows as a flow chart a possible neural network for use in a method according to the first aspect of the invention,

FIG. 2 shows as a flow chart a method according to the second aspect of the invention, and

FIG. 3 is a schematic illustration of a system for executing methods for predicting a value of a physical and/or chemical property, for design of a molecule and for training of a neural network according to the invention.

### BEST MODE FOR CARRYING OUT THE INVENTION

The present invention relates to a computer-implemented method for automated design of a molecule, preferably of an emitter molecule for use in an OLED, in particular of a TADF emitter, the method using a genetic algorithm and as inputs (200), a set of starting molecular structures including at least one molecular structure, a set of mutation rules for specifying allowed mutations for the genetic algorithm, a scoring function for a molecular structure based on one or more predicted values of the physical and/or chemical properties of the corresponding molecule, and a termination condition, the method providing as output, the molecular structure of the designed molecule, including the following steps: Providing (220) the set of starting molecular structures as a population of parent molecular structures to the genetic algorithm; generating (240) a population of offspring molecular structures partially or completely from the population of parent molecular structures using the genetic algorithm by mutating at least one member of the population using the genetic algorithm; predicting (260) one or more values of the physical and/or chemical properties of the molecules corresponding to the members of the population of offspring molecular structures and calculating a value for the scoring function for each member of the population of offspring molecular structures based thereon, wherein the one or more values of the physical and/or chemical properties are predicted using a neural network using a transformer with self-attention decay on molecular structures predicting one or more values of the physical and/or chemical properties of the members using the molecular structure as input; checking (270) if the termination condition is met and if not (210), generating (220) a new population of parent molecular structures consisting of at least one member of the population of offspring molecular structures having the most optimal value of the scoring function among the offspring molecular structures; iterating (210) these steps until the termination condition is met; and selecting (280) a molecular structure generated as an output.

### MODE FOR INVENTION

The object is achieved by the subject-matter according to the independent claims. Advantageous embodiments are defined in the dependent claims and the following description.

According to a first aspect of the present invention, a computer-implemented method for predicting a value of a physical and/or chemical property of a molecule can preferably be used for an automated design of a molecule.

In particular, this method for automated design of a molecule, preferably an emitter molecule and/or a host molecule for use in an OLED, in particular of a TADF (thermally activated delayed fluorescence) emitter, uses a genetic algorithm and as inputs a set of starting molecular structures including at least one molecular structure, a set of mutation rules for specifying allowed mutations for the genetic algorithm, a scoring function for a molecular structure based on one or more predicted values of physical and/or chemical properties of the corresponding molecule, and a termination condition. Furthermore, the method provides as output, the molecular structure of the designed molecule. It includes as steps providing the set of starting molecular structures as a population of parent molecular structures to the genetic algorithm, generating a population of offspring molecular structures partially or completely from the population of parent molecular structures using the genetic algorithm by mutating at least one member of the population using the genetic algorithm, and predicting one or more values of physical and/or chemical properties of the molecules corresponding to the members of the population of offspring molecular structures and calculating a value for the scoring function for each member of the population of offspring molecular structures based thereon. Here, the one or more values of physical and/or chemical properties are predicted by a neural network using a transformer model with self-attention decay on the molecular structure predicting one or more values of physical and/or chemical properties of the members using the molecular structure as input, wherein the machine-learning model preferably is designed as one of the models described further below. Afterwards, the termination condition is checked and in case it is not met, a new population of parent molecular structures consisting of at least one member of the population of offspring molecular structures having the most optimal (might be highest or lowest) value of the scoring function among the offspring molecular structures is generated.

The last steps (generating a population of offspring molecular structures, predicting physical and/or chemical properties of the molecules and calculating values for the scoring function, generating a new population of parent molecular structures) are iterated until the termination condition is met. Afterwards, one of the generated molecular structures is selected as output. This termination condition may be dependent on the value of the scoring function for the most optimal molecular structure in the current generation, but may also or additionally depend on the number of iterations (i.e., only a certain number of iterations are conducted). In particular, the termination condition may be defined as fulfilled if the value of the scoring function for at least one member of the population offspring structure is greater or smaller than or equal to a pre-defined value and/or a pre-defined number of iterations is reached.

In other words, the method optimizes a set of molecules (population) over a set of genetic optimization iterations (generations). In each generation, a set of parent molecules is selected based on a scoring function, for which the value of the scoring function (score) is to be maximized or minimized. The scoring function takes into account a set of physical and/or chemical properties that are estimated with the help of machine learning models. The selected molecules are then modified (mutated) by performing a random set of possible elementary modifications. Finally, for the next generation, this process is repeated until the termination condition, for example a certain value of the scoring function is met.

After each generation of new molecular structures, the molecular structures are assigned a scalar value, the score, also called fitness, based on the scoring function. This score describes how well the molecular structures matches the desired target criteria and is optimized by the algorithm by allowing high-scoring molecular structures to survive and produce offspring with a higher probability. For calculating the score, one or more values of physical and/or chemical properties (given as scalar values) are predicted using a neural network with a transformer with self-attention decay on molecular structures, wherein the molecular structures could for example be provided as SMILES strings. It is possible, that a user defines a scoring function (for example a linear ramp, a Gaussian shaped curve, or similar) by selecting a set of physical and/or chemical properties (features) that the user is interested in. Each of these features may then be calculated and mapped with a user-configurable scoring function to a feature score in a defined range, for example [0,1], that specifies how well the feature value fits the desired target value. Finally, individual feature scores may be combined, possibly via a user-configurable aggregation function, to a final score per molecular structure.

Preferably, mutating using the genetic algorithm is a fragment-based mutation and/or a string-based mutation. For fragment-based mutation, the molecular structures of molecules have to be represented as graphs of interconnected molecule fragments, which are added, removed, or replaced for a mutation. In contrast, a string-based mutation is based on string representing a molecular structure and applies elementary (pre-defined) transformations to the string representing the molecular structure at certain positions. For example, a string-based mutation can utilize SMARTS (SMILES arbitrary target specification). In this case, the string is a SMILES (simplified molecular input line entry specification) string, and SMARTS pattern matching can be used to find suitable positions for transformations.

In more detail, for fragment-based mutation, a molecular structure of a molecule is represented as a graph of interconnected molecule fragments. Here, a molecule fragment (also called fragment) is a representation of a molecular structure with a number of placeholder atoms (symbols used in the graph marking positions where other fragments can be linked to when mutating the fragment) that are connected to exactly one other atom of the fragment and that can be linked to placeholder atoms in different fragments. As placeholder atoms instead of purely abstract symbols, also atoms of otherwise not used elements, for example atoms of the actinide series (e.g., Am, Bk), can be used.

In general, a graph representing a molecular structure may be constructed by merging fragments along the linked placeholder atoms by adding a single bond between two atoms connected to the placeholder atoms in the link and removing the placeholder atoms. A graph consisting of fragments is referred to as a fragment graph. Therefore, also a single fragment is a fragment graph. A fragment graph is for example generated by connecting the following three fragments with two Am-Bk links as marked with double arrows:

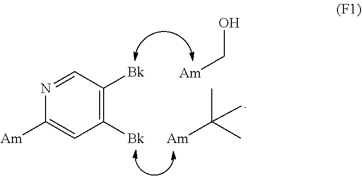

After removing the remaining Am atom, the molecule represented is the following:

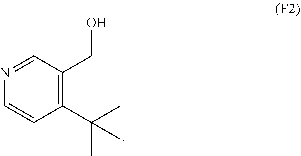

Fragment-based mutation can be configured by specifying a set of fragments, for example in the form of a SMILES string, with placeholder atoms as described above and defining for each fragment a positive weight that specifies the relative probability to pick this frequent in a mutation. Additionally, a set of connection rules may be specified for each placeholder atom type, i.e., a list of rules specifying for each placeholder atom type a list of placeholder atom types that this placeholder atom type can be connected to. For example, in case of Am, Bk, and U atoms acting as placeholder atoms in a set of fragments, a connection rule might be that Am atoms can only be connected to Am or Bk atoms and Bk atoms to Am or Bk atoms (which might be denoted as “(Am, Bk)”), but U atoms can only connect to other U atoms. The use of placeholder atoms makes it possible to allow only specific connections between certain fragments and/or sets of fragments.

For mutating a molecular structure using fragment-based mutation several operations are possible. Possible mutations include:
Addition of leaf fragment: A random fragment with a random free linking position is picked and a random fragment that can be connected to this linking position according to the connection rules is added and linked to the fragment graph. The fragment added is called a leaf fragment, since it is only connected to a single other fragment. For example:

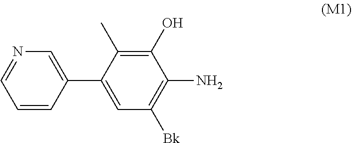

is mutated to

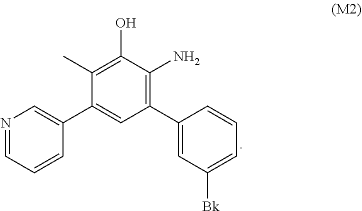

Removal of leaf fragment: A random fragment that is connected to only a single fragment (i.e., a leaf in the fragment graph) is removed from the fragment graph, for example:

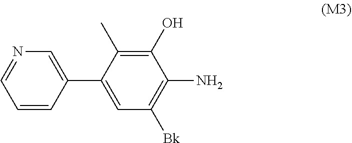

is mutated to

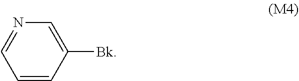

Addition of inner fragment: A random link (connected fragments) is picked, and a random fragment that can connect to the corresponding placeholder atoms is inserted at the link position, for example

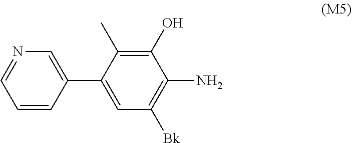

is mutated to

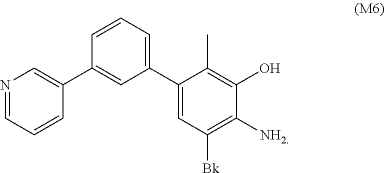

Removal of inner fragment: A random fragment that is connected to two fragments which can be connected according to the connection rules is removed from the fragment graph, for example:

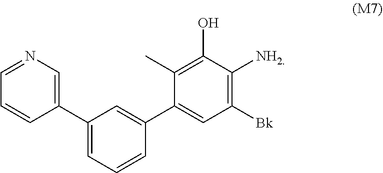

is mutated to

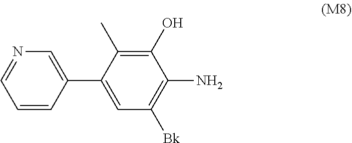

Replacement of fragment: A random fragment is replaced by a random fragment that has at least the amount of placeholder atoms of each type to replace the currently linked fragment (e.g., if the fragment to replace is connected at two Am and one Bk placeholder atoms, a fragment with at least two Am and one Bk placeholder atoms is chosen). The new fragment is linked to the fragment graph at random compatible positions while the current fragment is removed from the fragment graph. For example:

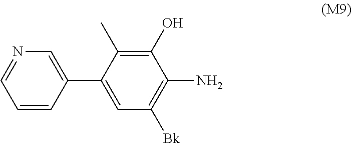

is mutated to

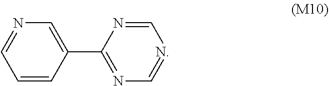 [SMILES: c1cncc(-c2ncncn2)c1]

Cross-over: It is also possible that with a defined probability a so-called “cross-over mutation” is performed instead of a normal mutation. A cross-over mutation uses two fragments, i.e., a second individual is sampled from the current pool of parent structures. The two parent molecules (first parent and second parent) are then combined by first splitting each parent molecule at a random link of the same type (e.g., between the same placeholder atom types) and then reconnecting compatible molecule parts from each parent. For example (the child molecule is generated by the encircled molecule parts of the first and second parent):

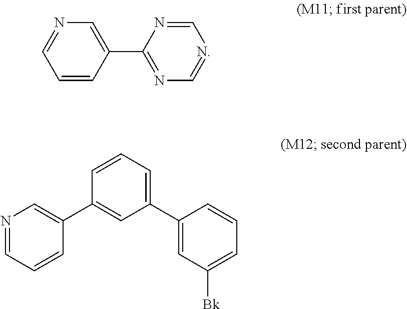

is combined to

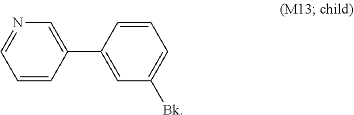

In all these operations, where a random choice is made, a weighted random choice is also possible instead of an equally distributed one. Typically, when mutating a molecular structure, one of these operations is picked at random or weighted random.

In the following, as an example, possible fragments are shown with Am, Bk, and U atoms acting as placeholder atoms (linker atoms):

Eight donor fragments with Am linker atoms:

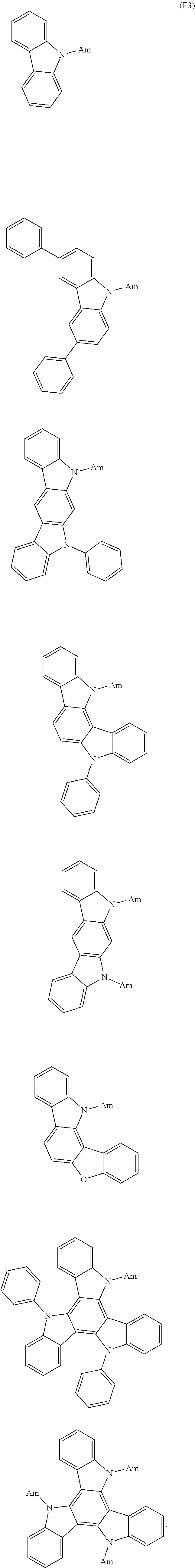

Four acceptor fragments with Bk linker atoms:

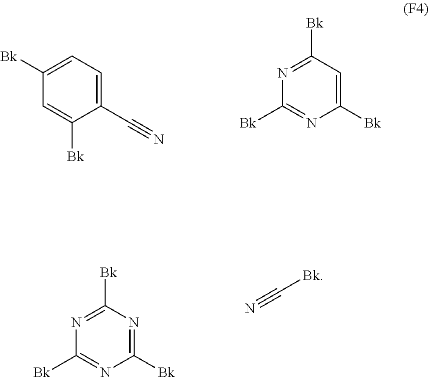

Two spacer fragments with U linker atoms:

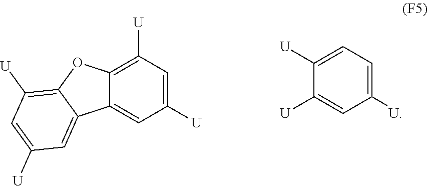

These fragments can be used in a method for automated design of a molecule according to the invention using fragment-based mutation and are particularly advantageous for designing of TADF emitters. In this case, connection rules for example can be (Am, Bk), (Am, U), (Bk, U), (U, U), i.e., donors can connect to acceptors or spacers, acceptors can connect to donors or spacers, and spacers can also connect to themselves.

An alternative to a fragment-based mutation is a string-based mutation, which is for example SMARTS-based. Instead of keeping track of a graph of fragments, a string-based mutation strategy represents a molecular structure as a single string, for example as a SMILES string. However, as in the case of the fragment-based mutation, this string may contain additional markers, for example in the form of isotope tags and/or placeholder atoms (also called linker atoms), for example from the actinide series, that are removed for output and scoring. These tags may be used to find specific locations introduced by previous mutations and thereby allow for the definition of fragment-based mutation with the string-based approach as well. Compared with the fragment-based approach, the string-based mutation approach allows for the definition of more fine-grained, elementary molecule manipulations consisting of: addition/removal of bonds, change of atom type/bond type, swapping atom types between neighboring atoms, addition/removal of a substructure at the edge of the molecule or between existing bonds, shifting of substructures connected to one atom to a neighboring atom, substitution of a set of atoms with another set of atoms. All in all, the string-based mutation can be seen as a more general variant of the fragment-based mutation.

Furthermore, it is advantageous for most applications, to use tournament selection and/or elitism for the genetic algorithm. To generate a new generation of parent molecular structures (parent selection) using tournament selection, a certain number of “tournaments” is performed by selecting a defined number of molecular structures of the current population of offspring molecular structures or (in the first iteration) starting molecular structures for each tournament at random. For each tournament, a certain number of the molecular structures with the highest score in the respective tournament are selected to form the set of parent molecular structures for the next generation, i.e., those molecular structures which are used for mutation. Elitism may be used additionally. Here, a defined number of best scoring molecular structures in the current population of offspring molecular structures or (in the first iteration) starting molecular structures is kept without mutation. Instead, they are directly copied to the next generation, i.e., they are automatically included in the next population of offspring molecular structures.

All in all, the best scoring molecular structures (from elitism) and the molecular structures generated (by mutation, for example using tournament selection) form the next population of parent molecular structures.

Preferably, for the prediction of the one or more values of the physical and/or chemical properties of the molecules corresponding to the members of the population of offspring molecular structures, a computer-implemented method for predicting a value of a physical and/or chemical property of a molecule, especially an emitter molecule and/or a host molecule for use in an OLED, in particular of a TADF (thermally activated delayed fluorescence) emitter, is used.

This prediction method uses as an input a molecular structure of the molecule as an atom-bond-graph, which is a graph including at least atoms and bonds of the molecular structure as nodes. In case no other node types are used, such a graph can be divided in two disjoint and independent sets (atoms and bonds) connected by edges. In that case it is a bipartite undirected graph. For example, the atom-bond-graph can be generated from a SMILES (simplified molecular input line entry specification) string representing the molecular structure examined. In the context of this invention, a molecular structure of a molecule is to be understood as information on the arrangement of the atoms of the molecule including at least the type of bonds between the atoms of the molecule as well as the element of each atom of the molecule.

Optionally, a so-called global node connected to all other nodes can be added when generating the atom-bond-graph. Such a global node can serve as a global (graph-level) attribute to transfer information and/or context that concerns the whole molecule to/from distant nodes (e.g., atoms and/or bonds as nodes) for the neural network used in the method. Therefore, it acts as a shortcut for exchanging global information between nodes, bypassing the need for information to travel along edges in multiple steps. Adding a global node often improves prediction performance and therefore is generally highly beneficial.

The method provides as output, the predicted value of the physical and/or chemical property (preferably, as a scalar value) and includes the following steps:
First, for each node of all n_{nodes} nodes of the atom-bond-graph a feature vector of dimension d_{features} is extracted, the feature vector including a node type (for which one-hot encoding can be used), the node type may for example be “atom”, “bond”, or “global”, and further data on the respective node at least in case of the node type being atom or bond, called node feature data. The node feature data may include for atoms the atomic number (for example as one-hot and/or as integer), a flag for being aromatic, a flag for being part of a ring structure, and/or the number of connected implicit hydrogen atoms. For bonds the node feature data may include the bond type (i.e., single, double, triple, aromatic). A feature vector x with dim x=d_{features} may have the form x=(node_{type}, featₐₜₒₘ, feat_{bond}), where node_{type} encodes the node type, featₐₜₒₘ the node feature data in case of an atom (atom features, which preferably are zero in case of other node types like bond and global), and feat_{bond} the node feature data in case of a bond (bond features, which preferably are zero in case of other node types like atom and global). The parameter node_{type} can be a one-hot encoding of the node type, which is one of atom, bond, or global. The use of a global node is optional. In case of the use of a global node, i.e., in case the atom-bond-graph includes a global node, the feature vector x may have the form x=(node_{type}, featₐₜₒₘ, feat_{bond}, feat_{global}), where feat_{global} encodes node feature data for the global node (global features, which preferably are zero in case of other node types like atom and bond). The node feature data might represent or include any parameter relevant for all other nodes.

For certain elements of the feature vector, it is advantageous to provide the same information in different formats, for example, the atomic number can be added to the feature vector both as integer and as one-hot. One-hot encoding typically makes it easier for the neural network used to treat a specific atomic number in a special way, but removes metric information, i.e., which atomic numbers are similar. For more continuous properties derived from the atomic number, for example the total molecular weight, it is more natural to process the atomic number directly, i.e., as integer.

All in all, n_{nodes} feature vectors are created, one for each node of the n_{nodes} nodes of the atom-bond graph. From these feature vectors, a feature matrix of dimension n_{nodes}×d_{features} is composed, which may have the form (x₁, x₂, x₃, . . . , xₙ_{_{nodes}}), with xᵢ being feature vectors. Also, a squared distance matrix D of dimension n_{nodes}×n_{nodes} is calculated based on and/or including distances between atoms and bonds of the molecular structure. The squared distance matrix is the n_{nodes}×n_{nodes} matrix with the (i, j)-element equal to 0 if i=j, and dᵢⱼ² if i≠j, where each node is assigned an index and i, j are representing such node indices and dᵢⱼ is the distance between the nodes i and j. Accordingly, the squared distance matrix includes information on distances dᵢⱼ between atoms and/or bonds of the molecular structure and can be based either on metric distances between nodes (derived from atom coordinates experimentally determined and/or simulated) or on abstract distances between nodes purely derived from the atom-bond-graph without information on atom coordinates. In case of the use of a global node, the squared distance matrix D might include values for distances between the global node and the other nodes (for example of the type atom and bond). The distance between a global node and another node is preferably an identical value c for all other nodes, i.e., dᵢⱼ2=c if either i or j represents the global node. The value c is preferably 0.

Finally, the feature matrix is fed into a trained neural network including a transformer model (transformer) as machine learning model as first described in Vaswani et al., “Attention Is All You Need”, 2017 while using the squared distance matrix D for self-attention decay to generate a prediction of the value of the physical and/or chemical property of the molecule.

Using a method according to the invention, physical and/or chemical properties such as a HOMO (highest occupied molecular orbital) energy level, a LUMO (lowest unoccupied molecular orbital) energy level, a singlet energy level, a triplet energy level, a singlet-triplet energy gap, an oscillator strength, a dipole moment, a photo-luminescent quantum yield, a delayed fluorescence lifetime, and/or a peak emission wavelength can be predicted.

The method uses a neural network which includes (as the transformer) a trained transformer-encoder stack with n_{layers}>1, preferably 6, transformer-encoder layers using the squared distance matrix D for self-attention decay, and a trained input encoder (including an input encoder layer) configured to generate an input matrix with dimension n_{nodes}×d_{model} as input for the trained transformer-encoder stack generated from the feature matrix of dimension n_{nodes}×d_{features}, where d_{model} is the dimension of the transformer model, for example d_{model}=128. Here, the trained transformer-encoder stack is configured to generate a matrix of dimension n_{nodes}×d_{model} as output while using the input matrix generated by the input encoder as input. Each of the n_{layers} transformer-encoder layers of the trained transformer-encoder stack after the first layer uses the output of the forgoing layer as input, which is also a matrix of dimension n_{nodes}×d_{model}. Applying the squared distance matrix D has the advantage of reintroducing distance information lost due to the step of generating the feature matrix.

Furthermore, the neural network may include a trained projection layer including an self-attention layer configured to generate (using the matrix generated by the trained transformer-encoder stack, i.e., its output, as input) a vector of dimension d_{model} as output, and finally, a trained multilayer perceptron (MLP) configured to generate the prediction of the value of the physical and/or chemical property (as target), for example in form of a scalar, from the output vector of the trained projection layer. Preferably, the MLP has one or two hidden layers and/or utilizes ReLU (Rectified Linear Unit) activation and/or dropout. It should be noted that several sets of projection layer plus MLP can be used in parallel, directed at different targets, i.e., at different physical and/or chemical properties. In other words, in the final layers of the neural network, the extracted information from the network is combined with an additional self-attention layer (in form of the projection layer) and a feed-forward network (the MLP) per target to predict a set of scalar quantities per molecular structure.

Preferably, the trained transformer-encoder stack consist of n_{layers} transformer-encoder layers with each having a trained multi-head decaying self-attention (i.e., a group of multiple, for example 4, decaying self-attention layers running in parallel), a trained feed-forward network (transformer feed-forward network; for example with a dimension of 512), two trained normalization layers (applying a function LayerNorm and preferably being layer normalizations according to Ba et al., “Layer Normalization”, 2016; one for the output of the transformer-encoder layer and one for the output of the feed-forward network), and two residual connections around the transformer-encoder layer and the feed-forward network (sublayers) allowing for an implementation of LayerNorm (x+sublayer (x)), where x is the input of the respective sublayer, as described in Vaswani et al. 2017 and section S7 (Solution 1) of Bratholm et al., “A community-powered search of machine learning strategy space to find NMR property prediction models”, 2020. It should be noted that the transformer makes no difference between atoms and bonds. Therefore, and to generate a representation for each atom-bond-graph having identical dimensions, a trained input encoder is used to transform the feature matrix consisting of feature vectors to a matrix of dimension n_{nodes}×d_{model}.

Also preferably, the transformer-encoder layers utilize an attention function for the self-attention decay that is given by

$$Attention ( Q , K , V ) = softmax ( \frac{{QK}^{T}}{\sqrt{{d}_{k}}} - γ D ) V , \qquad ( 1 )$$

where Q is a query matrix consisting of query vectors, K a key matrix consisting of key vectors, V a value matrix consisting of value vectors, dₖ the dimension of a key vector, D the squared distance matrix, y a trainable parameter, and softmax the softmax function (normalized exponential function) as described in Vaswani et al. 2017 and section S7 (Solution 1) of Bratholm et al., 2020. The matrices Q, K, V can be calculated using trained matrices from the input matrix of the respective group of self-attention layers of the transformer-encoder stack. In other words, Q, K, V are calculated from the input matrix of the current self-attention layer, which is the output of the input encoder for the first group of self-attention layers and the output of the previous self-attention layer otherwise, via a trainable linear transformation. Using the squared distance matrix D to modify the matrix within the argument of the softmax function reduces the interaction strength between pairs of faraway nodes of the atom-bond graph. The trainable decay parameter γ can be used to scale this influence of the squared distance matrix D and may be initialized as 1. A value of 0 for the parameter γ allows attention over the whole graph, whereas for γ=∞ attention is restricted to direct neighbor nodes in the graph. Thus, a distance-scaled self-attention is implemented.

In another advantageous embodiment, the method may use one or more additional input parameters as additional input for the trained multilayer perceptron. These additional input parameters may be physical and/or chemical parameters predicted with a method according to this invention and/or physical and/or chemical parameters predicted using a different method and/or measured physical and/or chemical parameters and/or parameters describing properties of the environment of the molecule under consideration. Such an approach allows for an increased flexibility and is also especially beneficial when training the neural network since typically available experimental or simulated data was generated under different conditions (for example, different emitter concentration and/or host material in case of OLED emitters). Therefore, to be able to use all available data from different experiments for training, the certain conditions of the experiments can be used as additional input features for the multilayer perceptron (i.e., as additional input parameters). After training, the additional input parameters can be set to the desired target conditions.

In a preferred embodiment of the method, the squared distance matrix D is calculated by using distances derived from the atom-bond-graph, i.e., not calculated using physical atom positions. Such a distance between two nodes of the atom-bond-graph is an abstract distance. Preferably, this distance is calculated by the edge distance (number of edges on the shortest path between two nodes) on the atom-bond-graph divided by two and rounded downward. For example, in case of the structure “C1-s4-C2-d5-O3” (s4 being a single, d5 a double bond, the C1, C2 carbon atoms, O3 an oxygen atom, the numbers representing node indices), the distance d between C1 and d5 is d (C1, d5)=floor (3/2)=1. Consistent with this definition, when using a global node, the distance between the global node and any other node may be defined as 0.

It was found that a squared distance matrix based on such abstract distances (and not physical distances) is sufficient for most applications, especially when simulating properties of emitter molecules for the use in OLEDs. Beneficially, this approach costs less calculation power than the use of real atom coordinates and requires less computer memory since integers can be used. Therefore, it is possible to simulate physical/chemical properties based on abstract distances extracted from an atom-bond-graph without having 3D information available. This also immensely simplifies the presented method, since no additional simulations are required for estimating atom positions.

In an especially beneficial embodiment of the method, a part of the trained neural network, for example the transformer of the trained neural network, was initialized using transfer learning. It was found that for initialization, parts of another trained neural network suitable for performing one of the methods presented above can be used when having trained this other neural network with suitable data, which might be more easily accessible. In particular, it is possible to pre-train the transformer and the input encoder using values of physical and/or chemical properties as targets, which can separately be simulated, for example using DFT (density functional theory). This allows for creating a large amount of training data for training of the transformer and the input encoder. Preferably, for training the final model, freshly initialized heads of the neural network, i.e., projection and MLP layers, are first trained with the pre-trained transformer and the input encoder frozen for a defined number of epochs with a constant learning rate. Afterwards, the transformer encoder can be unfrozen and fine-tuned together with the heads with a cosine learning rate schedule.

According to a further aspect of the invention, a system including at least one processor and a storage device is configured to execute a computer-implemented method according to one of the methods of the invention. The computer-implemented methods as presented above can be realized as computer programs being executed by such a system.

According to a another aspect of the present invention, a neural network for use in a method according to any of the computer-implemented methods presented above and including a transformer configured to use a squared distance matrix D for self-attention decay, one or more projection layers including an self-attention layer configured to generate a vector as output using a matrix generated by the transformer as input, and one or more multilayer perceptrons using the output of one of the one or more projection layers as input, can be trained with a method including the following steps: First, a data set including a molecular structure of a molecule and a value (as ground truth) of a physical and/or chemical property of the molecule is provided, where the physical and/or chemical property is assigned to one of the multilayer perceptrons.

The molecular structure of each molecule is converted to an atom-bond-graph including at least atoms of the molecular structure and bonds of the molecular structure as nodes. For each node of all n_{nodes} nodes of the atom-bond-graph, a feature vector of dimension d_{features} is extracted, the feature vector including a node type, the node type preferably being one of atom, bond, and global, and further data (node feature data) on the node in case of the node type being atom or bond. Afterwards, a feature matrix of dimension n_{nodes}×d_{features} composed of the extracted n_{nodes} feature vectors is generated. The squared distance matrix D of dimension n_{nodes}×n_{nodes} based on and/or including distances between atoms and bonds of the molecular structure is calculated. Finally, an output value of the multilayer perceptron assigned to the physical and/or chemical property is generated and the neural network adjusted based on comparing the output value of the multilayer perceptron to the value of the physical and/or chemical property assigned to the multilayer perceptron. For comparing the output with the ground truth typically a loss function is used. These steps can be repeated for a certain number of epochs with different data sets including a molecular structure of a molecule and a value of a physical and/or chemical property until the neural network has been sufficiently trained.

In an advantageous embodiment of the method, the neural network includes at least two multilayer perceptrons and the steps as described above are repeated for a different data set including the molecular structure of the molecule and a value for a different physical and/or chemical property, the different physical and/or chemical property being assigned to a different multilayer perceptron. This embodiment is based on the surprising finding that training for the main parts, especially the transformer, of the neural network as used in the methods according to the first and second aspects of this invention can be performed by using data as ground truth for different physical and/or chemical properties when using multiple multilayer perceptrons as separated outputs of the neural network, i.e., every physical and/or chemical property has to be assigned to a perceptron.

Similarly, a part of the neural network, for example the transformer, can be initialized using the corresponding part of another trained neural network suitable for use in a method according to any the first and second aspect of this invention. As already mentioned, it was found that for initialization, parts of another trained neural network can be used when having trained this other neural network with suitable data, which might be more easily accessible and/or generatable. This way, a large amount of training data can be generated. Preferably, for training the model after initialization using such a pre-trained part of another trained neural network, freshly initialized heads of the neural network including the multiplayer perceptrons are first trained with the pre-trained part for a defined number of epochs with a constant learning rate. Afterwards, the rest of the neural network can be unfrozen and fine-tuned together with the heads with a cosine learning rate schedule. For these training methods, a system including at least one processor and a storage device is used, i.e., at least some, preferably all of the steps of the described training methods are performed and/or supported by means of this system. In particular, the training methods can be computer-implemented methods.

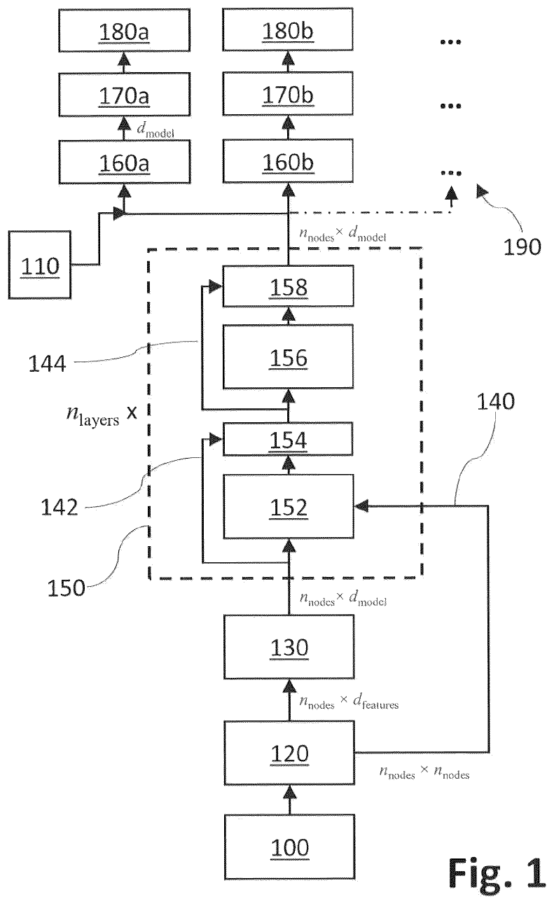

FIG. 1 shows in a schematic way a possible structure of software program including a neural network that may be used in a method according to the first aspect of the present invention for predicting a value of a physical and/or chemical property of a molecule. Additionally, this figure also illustrates the underlying method.

An atom-bond-graph 100 describing the molecular structure, for which a value of a physical and/or chemical property should be predicted, is provided (which is, for example, generated from a SMILES string) as input of this software program. This atom-bond-graph 100 is used by a graph feature extractor unit 120, which is a unit of the program which extracts for each node (atom, bond, global) of the atom-bond-graph 100 a feature vector which includes the node type of the node plus further data on the node in case of an atom or a bond and generates a feature matrix of dimension n_{nodes}×d_{features} consisting of nodes feature vectors with dimension d_{features}. Via an input encoder layer 130 this feature matrix is used as an input for a trained input encoder, which generates a matrix with dimension n_{nodes}×d_{model} which is used by an transformer-encoder stack consisting of n_{layers} transformer-encoder layers 150. Each of these layers 150 includes a trained multi-head decaying self-attention 152, a trained transformer feed-forward neural network 156, two layer normalizations 154, 158, and two residual connections 142, 144, which connect the input of the transformer-encoder layer with the first layer normalization 154 and the input of the feed-forward neural network 156 with the second layer normalization 158.

The multi-head decaying self-attention 152 consists of multiple parallel decaying self-attention layers, for example 4. The decaying self-attention can be implemented by using a function of the form as shown in formula (1).

The required matrix D used (arrow 140) is a squared distance matrix of dimension n_{nodes}×n_{nodes} calculated from the atom-bond-graph and may for example also be calculated and provided by the feature extractor unit 120 of the software.

The transformer encoder stack generates a matrix of dimensions n_{nodes}×d_{model} that is projected by one or more trained projection layers 160a, 160b, one for each target, including an self-attention layer to vectors of dimension d_{model}. In the figure, two projection layers 160a, 160b are shown, but more (190) are possible, dependent on the number of targets 180a, 180b. These vectors are used by MLPs 170a, 170b, which based on these vectors and optionally on one or more additional input parameters 110 (for example a property of a host molecule, a concentration, a solvent) finally calculate the targets 180a, 180b, i.e., the values of the physical and/or chemical properties as scalar values. As is the case for projection layers, the number of MLPs 170a, 170b depends on the number of targets 180a, 180b and can be more (190) than the two shown in the Figure.

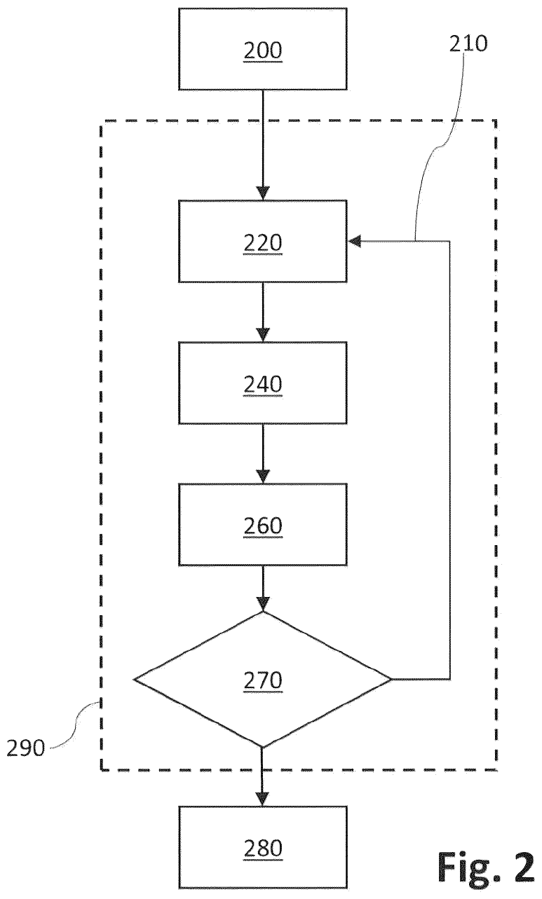

FIG. 2 shows as a flow chart for use in a method according to the second aspect of the invention. After initialization 200 of the genetic algorithm, which includes providing at least one molecular structure as stating molecular structure and the other required parameters (mutation rules, scoring function, termination condition), for example in form of a configuration file, in the parent selection step 220, the starting molecular structures are defined as parent molecular structures. Afterwards, in step 240, a fragment-based or string-based mutation is applied to produce a population of offspring molecular structures. In step 260, at least for these molecular structures at least one value of a physical and/or chemical property is predicted as a scalar value, for example using a method as shown in FIG. 1, and a value for a scoring function is calculated using this predicted value of the physical and/or chemical property. Afterwards the termination condition is checked in step 270, which might be dependent on the calculated value of the scoring function.

If the termination condition is not fulfilled (arrow 210), a new set of parent molecular structures are generated in step 220. For this, for example tournament selection can be used. The steps of generating a new parent generation (parent molecular structures) 220, generating a population of offspring molecular structures (using mutation) 240, predicting physical and/or chemical properties of the molecules and determining the score 260, and checking the termination condition 270 (these steps forming one iteration 290 of the process) are repeated until the termination condition is met. In case the termination condition is fulfilled, in step 280 one or more of the generated molecular structures are provided, which are typically the highest-scoring molecular structures generated.

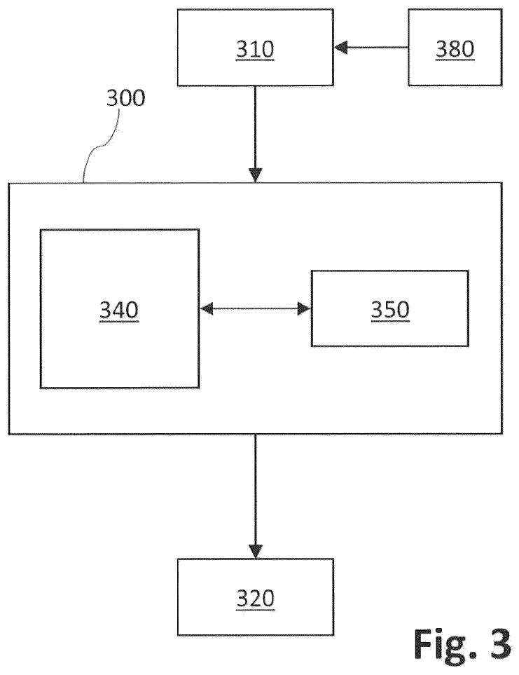

FIG. 3 shows a system 300 for executing methods for predicting a value of a physical and/or chemical property and/or for designing a molecule and/or for training of a neural network according to the invention. The system 300 includes at least one processor 340 connected to at least one storage device 350 and may include or may be a computer device, for example a personal computer or a mobile device. The system 300 or parts of the system may also be a distributed computing system, for example a cloud computing system.

A processor 340 of the system 300 may be for example a microprocessor, a central processing unit (CPU), an application-specific integrated circuit (ASIC), a graphics processing unit (GPU), a tensor processing unit (TPU), or a field programmable gate array (FPGA). A storage device 350 used may be for example a random access memory (RAM), a hard drive (HD), a solid-state drive (SSD), a flash memory, a compact disk (CD), or a digital video disk (DVD). The system may also include one or more display devices and one or more controllers, for example a keyboard, a computer mouse, and/or a touch screen.

The system 300 may be configured to execute a method for predicting a value of a physical and/or chemical property according to the invention and in this case may receive as input 310, for example provided by a human operator or a data bank 380, a molecular structure of the molecule in computer-readable form, for example as a SMILES string. This input 310 is converted by the system 300 to an atom-bond-graph 100 including atoms of the molecular structure and bonds of the molecular structure as n_{nodes} nodes of the atom-bond-graph 100. The system 300 may also be configured to receive 330 such an atom-bond-graph 100 as input 310 directly. Furthermore, the system 300 is also preferably configured to provide as output 320, the predicted value of the physical and/or chemical property after executing the method for predicting the value of the physical and/or chemical property according to the invention. Alternatively or additionally, the system 300 may also be configured to execute a method for automated design of a molecule according to the invention and in this case may receive as input 300, for example provided by a human operator or a data bank 380, a set of starting molecular structures, for example as a SMILES string, including at least one molecular structure, a set of mutation rules for specifying allowed mutations for a genetic algorithm, a scoring function for a molecular structure based on one or more predicted values of the physical and/or chemical properties of the corresponding molecule, and a termination condition. The system 300 may also be configured to provide as output 320, the molecular structure of the by the method designed molecule.

Alternatively or additionally, the system 300 may also be configured for executing a method for training of a neural network for use in a method according to the invention. In this case, the system 300 is designed to receive input training data, for example provided by a human operator or a data bank 380, including molecular structures of different molecules and values of physical and/or chemical properties of the molecules as input 310.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

1-16. (canceled)

17. A computer-implemented method,
wherein the computer-implemented method is for automated design of a molecule, and
wherein the computer-implemented method is utilizing a genetic algorithm and as inputs,
a set of starting molecular structures comprising at least one molecular structure,
a set of mutation rules for specifying allowed mutations for the genetic algorithm,
a scoring function for a molecular structure based on one or more predicted values of physical and/or chemical properties of the corresponding molecule, and
a termination condition, and
the computer-implemented method is providing as output, the molecular structure of the designed molecule,
the computer-implemented method comprising the following steps:
a) providing the set of starting molecular structures as a population of parent molecular structures to the genetic algorithm;
b) generating a population of offspring molecular structures partially or completely from the population of parent molecular structures utilizing the genetic algorithm by mutating at least one member of the population of parent molecular structures utilizing the genetic algorithm;
c) predicting one or more values of the physical and/or chemical properties of the molecules corresponding to the members of the population of offspring molecular structures and calculating a value for the scoring function for each member of the population of offspring molecular structures based thereon,
wherein the one or more values of the physical and/or chemical properties are predicted utilizing a neural network utilizing a transformer with self-attention decay on molecular structures predicting one or more values of the physical and/or chemical properties of the members utilizing the molecular structure as input;
d) checking whether the termination condition is met and if not, generating a new population of parent molecular structures consisting of at least one member of the population of offspring molecular structures having the most optimal value of the scoring function among the offspring molecular structures;
e) iterating the steps b, c, and d until the termination condition is met; and
f) selecting a molecular structure generated in the step b or any of its iterations as output.

18. The computer-implemented method according to claim 17, wherein the termination condition is met when the scoring function for at least one member of the population of offspring molecular structure is greater or smaller than or equal to a pre-defined value and/or a pre-defined number of iterations of the steps b, c, d is reached.

19. The computer-implemented method according to claim 17, wherein the mutating utilizing the genetic algorithm is a fragment-based mutation and/or a string-based mutation.

20. The computer-implemented method according to claim 17, wherein the genetic algorithm utilizes tournament selection and/or elitism.

21. The computer-implemented method according to claim 17, wherein for the prediction of the one or more values of the physical and/or chemical properties, a computer-implemented method for predicting a value of a physical and/or chemical property of a molecule is utilized, and
wherein
the computer-implemented method for predicting the value of the physical and/or chemical property of the molecule utilizes as input, a molecular structure of the molecule as an atom-bond-graph comprising at least atoms of the molecular structure and bonds of the molecular structure as nodes, and
the computer-implemented method for predicting the value of the physical and/or chemical property of the molecule provides as output, the predicted value of the physical and/or chemical property,
the computer-implemented method for predicting the value of the physical and/or chemical property of the molecule comprising the following steps:
a) extracting for each node of all n_{nodes} nodes of the atom-bond-graph a feature vector of dimension d_{features}, the feature vector comprising a node type, the node type being one of atom, bond, and global, and further data on the node in case of the node type being atom or bond;
b) generating a feature matrix of dimension n_{nodes}×d_{features} composed of the extracted n_{nodes} feature vectors;
c) calculating a squared distance matrix D of dimension n_{nodes}×n_{nodes} based on and/or comprising distances between atoms and bonds of the molecular structure; and
d) applying a trained neural network comprising a transformer utilizing the squared distance matrix D for self-attention decay on the feature matrix to generate a prediction of the value of the physical and/or chemical property of the molecule,
wherein the trained neural network comprises:
a trained input encoder configured to generate an input matrix with dimension n_{nodes}×d_{model} from the feature matrix of dimension n_{nodes}×d_{features}, where d_{model} is the dimension of a transformer model,
as the transformer a trained transformer-encoder stack with n_{layers}>1 transformer-encoder layers utilizing the squared distance matrix D for self-attention decay, wherein the trained transformer-encoder stack is configured to generate a matrix of dimension n_{nodes}×d_{model} as output utilizing the input matrix of the trained input encoder as input,
a trained projection layer comprising a self-attention layer configured to generate a vector of dimension d_{model} as output utilizing the matrix generated by the trained transformer-encoder stack as input, and
a trained multilayer perceptron configured to generate the prediction of the value of the physical and/or chemical property from the output vector of the trained projection layer.

22. The computer-implemented method according to claim 21, wherein each of the n_{layers} transformer-encoder layers comprises a trained multi-head decaying self-attention, a trained feed-forward network, two trained layer normalizations, and two residual connections.

23. The computer-implemented method according claim 21, wherein an additional input parameter is utilized as an additional input for the trained multilayer perceptron.

24. The computer-implemented method according to claim 21, wherein the physical and/or chemical property is at least one selected from among a highest occupied molecular orbital energy level, a lowest unoccupied molecular orbital energy level, a singlet energy level, a triplet energy level, a singlet-triplet energy gap, an oscillator strength, a dipole moment, a photo-luminescent quantum yield, a delayed fluorescence lifetime, and a peak emission wavelength.

25. The computer-implemented method according to claim 21, wherein the squared distance matrix D is calculated by utilizing abstract distances derived from the atom-bond-graph.

26. The computer-implemented method according to claim 21, wherein a part of the trained neural network is initialized by another transformer of another trained neural network suitable for performing the computer-implemented method for predicting the value of the physical and/or chemical property of the molecule.

27. A method for training,
wherein the method is for training of a neural network for utilization in the computer-implemented method according to claim 21, and
wherein the method utilizes a system comprising at least one processor and a storage device, and the neural network comprises a transformer configured to utilize a squared distance matrix D for attention decay, one or more projection layers comprising an attention layer configured to generate a vector as output utilizing a matrix generated by the transformer as input, and one or more multilayer perceptrons utilizing the output of one of the one or more projection layers as input,
the method comprising the following steps:
a) providing a data set comprising a molecular structure of a molecule and a value of a physical and/or chemical property of the molecule, where the physical and/or chemical property is assigned to one of the multilayer perceptrons;
b) converting the molecular structure of each molecule to an atom-bond-graph comprising at least atoms of the molecular structure and bonds of the molecular structure as nodes;
c) extracting for each node of all n_{nodes} nodes of the atom-bond-graph a feature vector of dimension d_{features}, the feature vector comprising a node type, the node type being one of atom, bond, and global, and further data on the node in case of the node type being atom or bond;
d) generating a feature matrix of dimension n_{nodes}×d_{features} composed of the extracted n_{nodes} feature vectors;
e) calculating the squared distance matrix D of dimension n_{nodes}×n_{nodes} based on and/or comprising distances between atoms and bonds of the molecular structure;
f) generating an output value of the multilayer perceptron assigned to the physical and/or chemical property; and
g) adjusting the neural network based on comparing the output value of the multilayer perceptron to the value of the physical and/or chemical property assigned to the multilayer perceptron.

28. The method according to claim 27, wherein the neural network comprises at least two multilayer perceptrons, and wherein the steps a to g are repeated for a different data set comprising the molecular structure of the molecule and a value of a different physical and/or chemical property, the different physical and/or chemical property being assigned to a different multilayer perceptron.

29. The method according to claim 27, wherein the steps a to g are repeated for a different data set comprising the molecular structure of a different molecule and a value of the physical and/or chemical property of the different molecule.

30. The method according to claim 27, wherein a part of the neural network is initialized utilizing another transformer of another trained neural network for utilization in the computer-implemented method.

31. A system comprising a processor and a storage device, wherein the system is configured to execute the computer-implemented method according to claim 17.

32. A computer program having a program code for performing the computer-implemented method according to claim 17 when the computer program is executed by a system comprising a processor and a storage device.

33. A system comprising a processor and a storage device, wherein the system is configured to execute the computer-implemented method according to claim 21.

34. A system comprising a processor and a storage device, wherein
the system is configured to execute the method according to claim 27.

35. A computer program having a program code for performing the computer-implemented method according to claim 21 when the computer program is executed by a system comprising a processor and a storage device.

36. A computer program having a program code for performing the method according to claim 27 when the computer program is executed by a system comprising a processor and a storage device.
